(19) United States
(12) Patent Application Publication
(10) Pub. No.: US 2012/0029174 A1
(43) Pub. Date: Feb. 2, 2012

(54) METHODS FOR PRODUCING SUBSTANTIALLY HOMOGENEOUS HYBRID OR COMPLEX N-GLYCANS IN METHYLOTROPHIC YEASTS

(75) Inventors: Nico Callewaert, Nevele-Hansbeke (BE); David A. Wiersma, Tucson, AZ (US)

(73) Assignees: VIB, VZW, Zwijnaarde (BE); Research Corporation Technologies, Inc., Tucson, AZ (US); Universiteit Gent, Gent (BE)

(21) Appl. No.: 13/241,543

(22) Filed: Sep. 23, 2011

Related U.S. Application Data
(62) Division of application No. 12/906,237, filed on Oct. 18, 2010.
(60) Provisional application No. 61/252,283, filed on Oct. 16, 2009.

(51) Int. Cl. C07K 14/39; C12P 21/00

(52) U.S. Cl. 530/395; 435/69.1

(57) ABSTRACT

The present invention provides methods for effectively and efficiently converting methylotrophic yeast's heterogeneous high mannose-type N-glycosylation to mammalian-type N-glycosylation by disruption of an endogenous glycosyltransferase gene (OCH1) and step-wise introduction of heterologous glycosidase and glycosyltransferase activities. Each engineering step includes a number of stages: transformation with an appropriate vector, cultivation of a number of transformants, performance of sugar analysis and heterologous protein expression analysis, and selection of a desirable clone. The selected clone is then subjected to the next engineering step.

### CROSS REFERENCE TO RELATED APPLICATION

This application is a divisional of U.S. patent application Ser. No. 12/906,237, filed Oct. 18, 2010, which claims the benefit of priority from U.S. Provisional Application No. 61/252,283, filed on Oct. 16, 2009, the entire content of which is incorporated herein by reference.

### FIELD OF THE INVENTION

This invention generally relates to genetic engineering of methylotrophic yeast. More specifically, the invention relates to converting methylotrophic yeast's heterogeneous high mannose-type N-glycosylation to mammalian-type N-glycosylation. Engineering methods, engineered strains, and glycoproteins produced from the engineered yeast strains are provided. The system may be used to generate variously glycosylated forms of a parent protein, such as biosimilars of therapeutic proteins.

### BACKGROUND OF THE INVENTION

Yeasts are widely used both by industrial and academic research laboratories for the production of heterologous proteins. Especially the methylotrophic yeast Pichia pastoris is extensively used as protein production platform. The popularity of this particular yeast is attributable to its ability to produce foreign proteins at high levels, the simplicity of techniques needed for its genetic manipulation, and its capacity to perform many eukaryotic co- and post-translational modifications, including N-glycosylation.

However, therapeutic glycoproteins intended for parenteral use in humans are so far typically produced in mammalian cells because of the ability of these cells to modify proteins with mammalian complex-type N-glycan structures. Yeasts are unfavorable in this respect, because they modify glycoproteins with non-human high mannose-type N-glycans. These structures drastically reduce in vivo protein half-life, may be immunogenic in man, and hamper downstream processing as a result of extreme heterogeneity.

N-glycosylation is the attachment of oligosaccharides to specific asparagine residues within the consensus sequence Asn-X-Ser/Thr. Briefly, in eukaryotes, this process occurs co-translationally and the central step takes place at the luminal side of the ER membrane, involving the transfer of a Glc₃Man₉GlcNAc₂ oligosaccharide to nascent polypeptide chains. This precursor structure is then further modified by a series of glycosidases and glycosyltransferases. The initial processing reactions take place in the ER. Following the removal of the three glucose residues by glucosidase I and II, one specific terminal α-1,2-mannose is removed by mannosidase I. These reactions are well conserved between most lower and higher eukaryotes. At this point, correctly folded Man₈GlcNAc₂ N-glycosylated proteins exit from the ER to the Golgi complex where the glycans undergo further species- and cell type-specific processing.

In higher eukaryotes, the Man₈GlcNAc₂ structures coming from the ER are further trimmed by several α-1,2-mannosidases. The resulting Man₅GlcNAc₂ N-glycans are subsequently modified by the addition of a β-1,2-linked GlcNAc residue in a reaction catalyzed by GlcNAc transferase I (GnT-I), leading to the formation of “hybrid-type” N-glycans. Upon removal of two mannoses by mannosidase II (Man-II), a second β-1,2-GlcNAc is added by GnT-II. Glycans with the resulting structure in which both core-α-mannose residues are modified by at least one GlcNAc residue, are called “complex type” N-glycans. The addition of galactose and sialic acid residues is catalyzed by galactosyltransferases and sialyltransferases, respectively. Additional branching can be initiated by GnT-IV, GnT-V, and GnT-VIs.

In contrast to higher eukaryotes, N-glycan diversity in yeast is typically generated by the addition of mannose and mannosylphosphate residues. Yeasts do not further trim the Man₈GlcNAc₂ glycans that arrive at the Golgi from the ER. Instead, these structures are modified by the addition of an α-1,6-mannose residue (indicated in red in FIG. 1a) to the α-1,3-mannose of the trimannosyl core, a reaction catalyzed by Och1p. This “initiating mannose” is then further elongated by several (phospho)mannosyltransferases. The resulting mannan-type structures consist of a backbone of up to several dozen α-1,6-mannoses with short side branches, collectively known as the “outer chain”. These N-glycans are often referred to as hyperglycosyl- or hypermannosyl-type structures.

The advent of biosimilars, therapeutic biologics similar but not identical to innovator therapeutic proteins, requires the development of systems for the generation of candidate therapeutic proteins similar to innovator products. Therapeutic proteins with variant N-glycosylation are one type of biosimilar therapeutic proteins. These variants can be made in select engineered Pichia strains and assayed to determine if they meet criteria for new innovator products or biosimilars.

### SUMMARY OF THE INVENTION

In one aspect, the present invention provides a highly efficient and effective engineering method to convert methylotrophic yeast's heterogeneous high mannose-type N-glycosylation to mammalian-type N-glycosylation (hybrid and complex-type structures). The present method involves disruption of an endogenous glycosyltransferase gene (OCH1) and step-wise introduction of appropriately localized heterologous glycosidase and glycosyltransferase activities, wherein each engineering step is comprised of transformation with an appropriate vector, cultivation of a number of transformants, analysis of the N-glycans of glycoproteins and expression of the heterologous glycoprotein of interest produced from each of the transformants, and selection of a desirable clone based on the analysis that produces the heterologous glycoprotein of interest with substantially homogenous N-glycans. If desired, the selected clone can be further engineered by repeating the procedure with the next vector in the engineering pathway.

Therefore, it is possible to produce a heterologous glycoprotein in a methylotrophic yeast strain engineered in accordance with the present invention, wherein the N-glycans on the heterologous protein are substantially homogeneous and are characterized by a predominant N-glycan structure.

Accordingly, in another aspect, the present invention provides an engineered methylotrophic yeast strain, which produces a heterologous protein bearing a predominant N-glycan structure selected from one of M5, GnM5, GalGnM5, GalGnM3, GnM3, Gn2M3, or Gal2Gn2M3.

In still another embodiment, the invention provides a panel of engineered methylotrophic yeast strains, each strain in the panel producing a heterologous protein bearing a predominant N-glycan structure, and the predominant N-glycan structures in the panel are M5, GnM5, GalGnM5, GalGnM3, GnM3, Gn2M3, and Gal2Gn2M3, respectively.

In a further aspect, the present invention provides a preparation of a glycoprotein having a predominant N-glycan structure selected from one of M5, GnM5, GalGnM5, GalGnM3, GnM3, Gn2M3, or Gal2Gn2M3.

In still a further aspect, the present invention provides a panel of preparations of a glycoprotein having a predominant N-glycan structure, wherein the predominant N-glycan structures for the panel are M5, GnM5, GalGnM5, GalGnM3, GnM3, Gn2M3, and Gal2Gn2M3, respectively.

In still a further aspect, the present invention provides a system for selection of a candidate biosimilar therapeutic protein from a panel of preparations of a glycoprotein having a predominant N-glycan structure, wherein the system includes engineering Pichia to produce several candidate N-glycan variants of a therapeutic recombinant protein, and assaying selected properties of the variant proteins to select the version that best meets certain pre-established criteria.

### BRIEF DESCRIPTION OF THE DRAWINGS

FIGS. 1a-1d: Glycoengineering strategy overview.

FIG. 1a: Schematic outline of the procedure for engineering the N-glycosylation pathway of P. pastoris using GlycoSwitch plasmids. Each engineering step results in the introduction of one glycosidase or glycosyltransferase activity in the Pichia ER or Golgi complex. The construction of a strain that modifies its glycoproteins with biantennary complex-type N-glycans (e.g. Gal2Gn2M3) with terminal galactose requires the introduction of five GlycoSwitch plamids. In all figures in this figure, the graphical representation for glycans as advised by the Consortium for Functional Glycomics is used: green circle: mannose, blue circle: glucose, blue square: N-acetylglucosamine, yellow circle: galactose. Only here in panel 1a, red circles are used to emphasize the α-1,6-linked polymannosyl backbone of yeast-type hypermannosylated glycans, of which the synthesis is abolished by inactivation of the OCH1 gene.

FIG. 1b: Many glycosidases and glycosyltransferases are type II membrane proteins. Their N-terminal region (cytosolic tail, transmembrane domain and part of the luminal ‘stem’ region) is responsible for correct subcellular localization. Proper targeting of each introduced glycosylation enzyme was achieved by fusing its catalytic domain (shown in red) with the amino-terminus of a yeast protein with a known subcellular localization (shown in green). Consequently, the introduced enzymes are in fact hybrid proteins with a yeast N-terminal localization domain.

FIG. 1c: Upon digestion of pGlycoSwitchM8 with BstBI and transformation in P. pastoris, this construct integrates at the OCH1 locus. This results in a short OCH1 fragment that does not result in the synthesis of a functional Och1 protein and a promotorless fragment that cannot give rise to a functional protein.

FIG. 1d: Each engineering step consists of four stages: 1) transformation with the appropriate GlycoSwitch vector; 2) small-scale cultivation of a number of transformants; 3) N-glycan analysis; 4) and heterologous protein expression analysis. If desired, the best clone in terms of N-glycan profile and protein expression level can then be further engineered by repeating the procedure with the next GlycoSwitch vector in line.

FIG. 2. SDS-PAGE analysis of medium proteins from mIL-10-producing strains. Strains were grown according to Steps 23-29 in the protocol. Proteins produced by equivalent amounts of yeasts cells were TCA-precipitated and loaded on gel. The predominant protein produced by these strains is mIL-10 (indicated with an arrow). The hyperglycosylation of the GS115mL10-produced protein is clearly visible. A small amount of non-glycosylated mIL-10 was produced by each strain. Proteolytic degradation of mIL-10 seemed to increase with further engineering of the strains. Glycoengineering did not severely decrease mIL-10 yields. GS115 ctrl=GS115 wild type strain not producing any heterologous protein.

FIGS. 3a-3b: DSA-FACE profiles for mIL-10. FIG. 3a: N-glycan profiles of the proteins present in unpurified growth medium after small-scale (Steps 23-29) cultivation of these strains; FIG. 3b: DSA-FACE N-glycan profiles of mIL-10 purified from 250 ml shake flask cultures. Electropherograms 1a and b show the results for a malto-dextrose reference. Electropherograms 2 through 8 show the results for N-glycans, as follows: Electropherogram 2b, GS115mL-10 (typical wild type P. pastoris profile); Electropherograms 3a and b, M5mL-10 (the predominant peak is Man₅GlcNAc₂); Electropherograms 4a and b, GnM5mL-10 (the predominant peak is GlcNAcMan₅GlcNAc₂); Electropherograms 5a and b, GalGnM5mL-10 (the predominant peak is GalGlcNAcMan₅GlcNAc₂); Electropherograms 6a and b, GalGnM3mL-10 (the main peaks are GalGlcNAcMan₃GlcNAc₂ and GalGlcNAcMan₄GlcNAc₂); Electropherograms 7a and b, Gal2Gn2M3mL-10 (the main peak is Gal₂GlcNAc₂Man₃GlcNAc₂); Electropherograms 8a and b, reference N-glycans from bovine RNase B (Man₅₋₉GlcNAc₂ [M5-M9]).

FIGS. 4a-4b: DSA-FACE and SDS-PAGE analysis of medium proteins from mGM-CSF-producing strains. (4a) N-glycan profiles of the proteins present in unpurified growth medium after small-scale cultivation of these strains (according to Steps 23-29 in the protocol). Electropherogram 1 shows the results for a malto-dextrose reference. Electropherograms 2 through 8 show the results for N-glycans of the glycoengineered expression strains, as follows: Electropherogram 2, GS115mGM-CSF (typical wild type P. pastoris profile); Electropherogram 3, M5mGM-CSF (the predominant peak is Man₅GlcNAc₂); Electropherogram 4, GnM5mGM-CSF (the predominant peak is GlcNAcMan₅GlcNAc₂); Electropherogram 5, GalGnM5mGM-CSF (the predominant peak is GalGlcNAcMan₅GlcNAc₂); Electropherogram 6, GalGnM3mGM-CSF (the main peaks are GalGlcNAcMan₃GlcNAc₂ and GalGlcNAcMan₄GlcNAc₂); Electropherogram 7, Gal2Gn2M3mGM-CSF (the main peak is Gal₂GlcNAc₂Man₃GlcNAc₂); Electropherogram 8, reference N-glycans from bovine RNase B (Man₅₋₉GlcNAc₂ [M5-M9]). (4b) Proteins produced by equivalent amounts of yeast cells (˜30×10⁷; corresponds to an OD₆₀₀ of ˜15) were TCA-precipitated and loaded on a 15% SDS-PAGE gel. Native samples are indicated with “−”; samples deglycosylated with PNGase F are indicated with “+”. The band marked with an asterisk is PNGase F. The arrow indicates non-N-glycosylated mGM-CSF. These gels indicate that glycoengineering process did not severely decrease mGM-CSF yields.

FIGS. 5a-5b: DSA-FACE and SDS-PAGE analysis of medium proteins from mIL-22-producing strains. (5a) N-glycan profiles of the proteins present in unpurified growth medium after small-scale cultivation of these strains (according to Steps 23-29 in the protocol). Electropherogram 1 shows the results for a malto-dextrose reference. Electropherograms 2 through 8 show the results for N-glycans of the glycoengineered expression strains, as follows: Electropherogram 2, GS115mL-22 (typical wild type P. pastoris profile); Electropherogram 3, M5mL-22 (the predominant peak is Man₅GlcNAc₂); Electropherogram 4, GnM5mL-22 (the predominant peak is GlcNAcMan₅GlcNAc₂); Electropherogram 5, GalGnM5mL-22 (the predominant peak is GalGlcNAcMan₅GlcNAc₂); Electropherogram 6, GalGnM3mL-22 (the main peaks are GalGlcNAcMan₃GlcNAc₂ and GalGlcNAcMan₄GlcNAc₂); Electropherogram 7, Gal2Gn2M3mL-22 (the main peak is Gal₂GlcNAc₂Man₃GlcNAc₂); Electropherogram 8, reference N-glycans from bovine RNase B (Man₅₋₉GlcNAc₂ [M5-M9]). (5b) Proteins produced by equivalent amounts of yeast cells (˜30×10⁷; corresponds to an OD600 of ˜15) were TCA-precipitated and loaded on a 15% SDS-PAGE gel. Native samples are indicated with “−”; samples deglycosylated with PNGase F are indicated with “+”. The band marked with an asterisk is PNGase F. The smear marked with “#” is believed to be the result of interfering endogenous mannosyltransferases and incomplete processing by the introduced enzymes. These gels indicate that glycoengineering did not severely decrease mIL-22 yields.

### DETAILED DESCRIPTION OF THE INVENTION

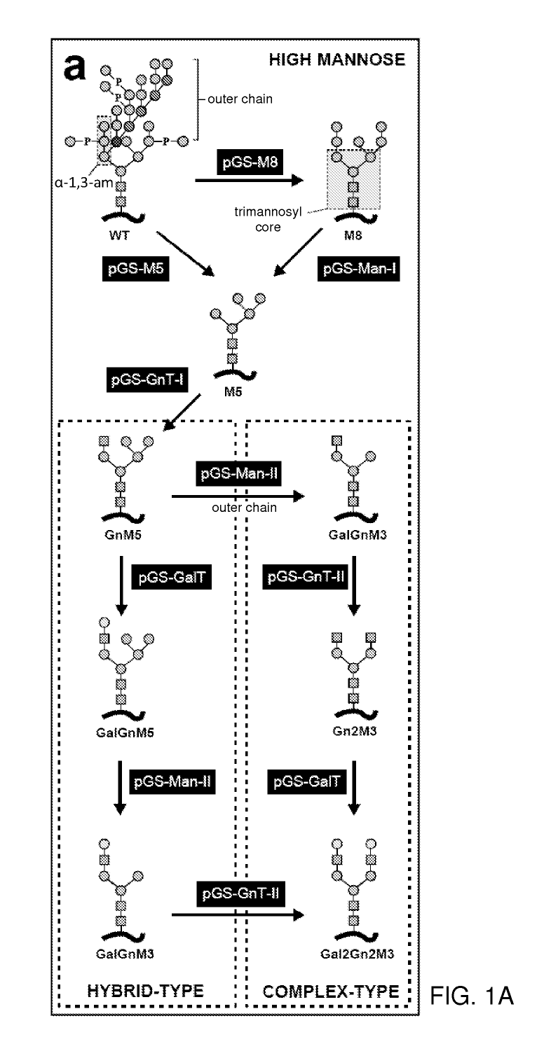

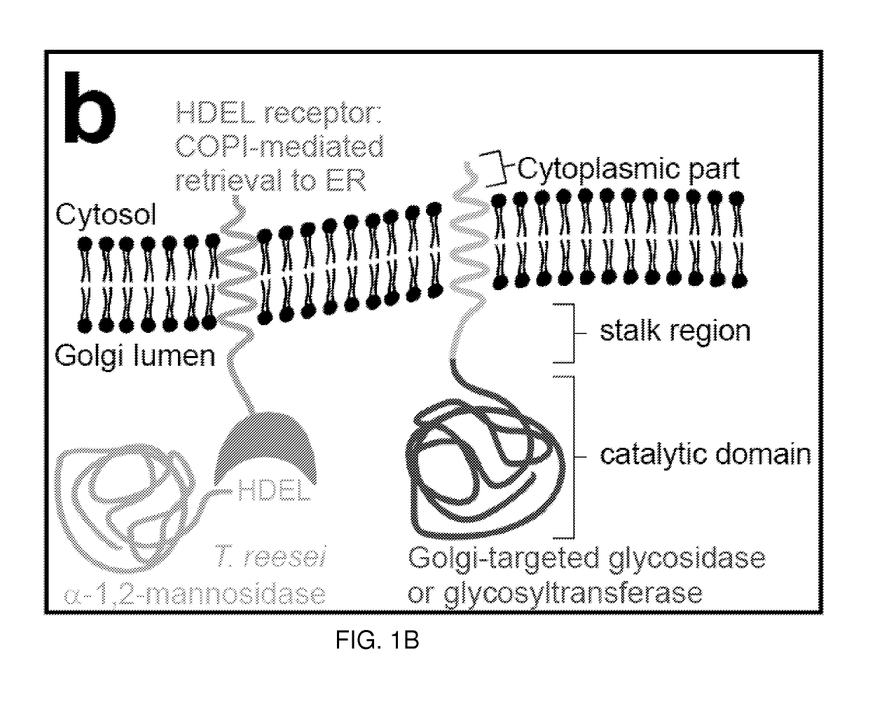

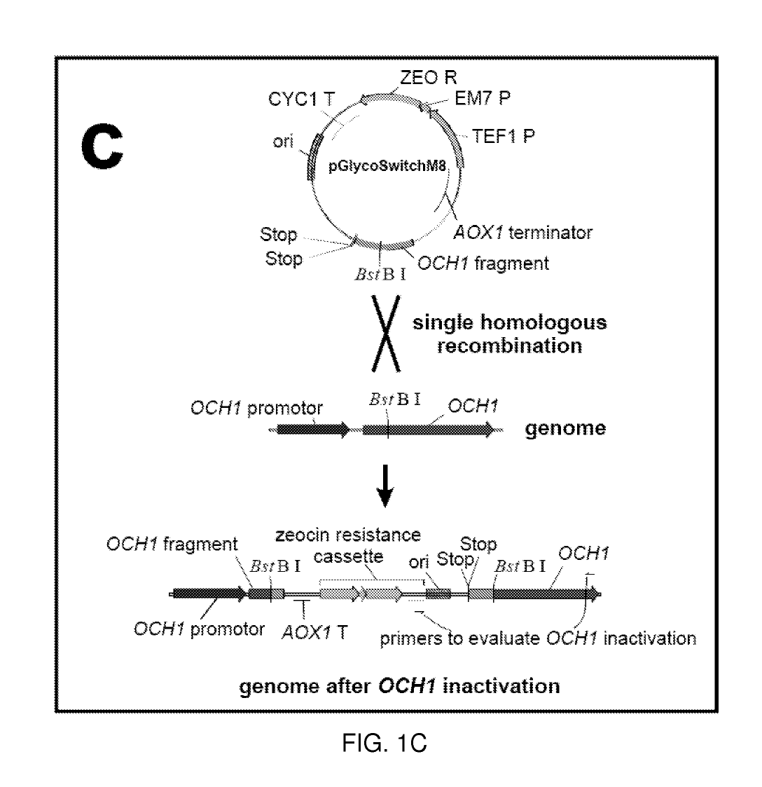

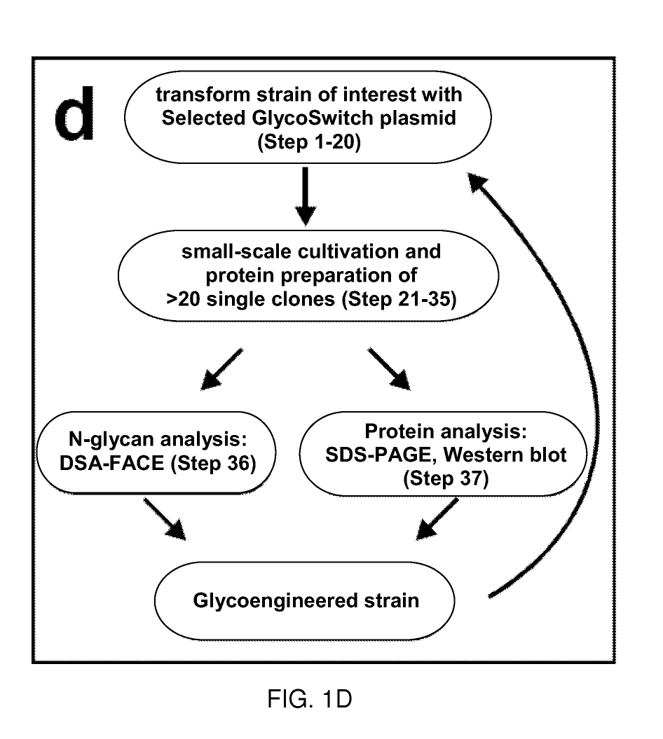

The present invention provides a highly efficient and effective engineering method to convert methylotrophic yeast's heterogeneous high mannose-type N-glycosylation to mammalian-type N-glycosylation (hybrid and complex-type structures). The present method involves disruption of an endogenous glycosyltransferase gene (OCH1) and step-wise introduction of appropriately localized heterologous glycosidase and glycosyltransferase activities (Table 1). Each engineering step includes a number of stages: transformation with an appropriate vector, cultivation of a number of transformants, performance of N-glycan analysis and heterologous protein expression analysis, and selection of a desirable clone based on the analysis. If desired, the selected clone can be further engineered by repeating the procedure with the next vector in the engineering pathway. An outline of the procedure for engineering the N-glycosylation pathway of P. pastoris is provided in FIG. 1a.

The unique glycoengineering strategy of the present invention, described below in more details, provides a surprisingly high engineering efficiency at each step in shake flask cultures of methylotrophic yeast. In other words, based on the glycoengineering strategy described herein, it is possible to obtain a heterologous glycoprotein in a methylotrophic yeast strain engineered in accordance with the present invention, wherein the N-glycans on the heterologous protein are substantially homogeneous and are characterized by a predominant engineered N-glycan structure or glycoform. Using appropriate criteria these homogenous N-glycan forms of the protein can be assayed and biosimilar forms of the protein selected.

By “substantially homogeneous” N-glycans it is meant that given a preparation containing a population of a particular glycoprotein of interest, at least 50%, 60%, 75%, 80%, 85%, 90% or even 95% of the N-glycans on the protein molecules within the population are the same.

By “predominant N-glycan structure” or “predominant glycoforms” it is meant a specific N-glycan structure or glycoform of (i.e., attached to) a glycoprotein represents the greatest percentage of all N-glycan structures or glycoforms of the glycoprotein, which is at least 2× (two fold), 3×, 4×, 5×, 7.5×, 10× the percentage value of any other N-glycan structure or glycoforms. In certain specific embodiments, a predominant glycoform accounts for at least 30%, 40%, 50%, 60%, 70%, 80%, 90% or 95% or greater of the population of all glycoforms of the glycoprotein.

Specific desirable glycoforms or N-glycan structures which can be generated in accordance with the glycoengineering strategy of the present invention includes M8 (Man₈GlcNAc₂), M5 (Man₅GlcNAc₂), GnM5 (GlcNAcMan₅GlcNAc₂), GalGnM5 (GalGlcNAcMan₅GlcNAc₂), GalGnM3 (GalGlcNAcMan₃GlcNAc₂), GnM3 (GlcNAcMan₃GlcNAc₂), Gn2M3 (GlcNAc₂Man₃GlcNAc₂), and Gal2Gn2M3 (Gal₂GlcNAc₂Man₃GlcNAc₂), the structures of which are depicted in FIG. 1a.

A strain that generates M8 as the predominant N-glycan structure is also referred hereto as an M8 strain. Similarly, a strain that generates M5, GnM5, GalGnM5, GalGnM3, GnM3, Gn2M3, and Gal2Gn2M3, respectively, as the predominant N-glycan structure, is also referred hereto as an M5, GnM5, GalGnM5, GalGnM3, GnM3, Gn2M3, and Gal2Gn2M3 strain, respectively.

The glycoengineering method provided by the present invention involves disruption of an endogenous glycosyltransferase gene (OCH1) in a methylotrophic yeast strain and a sequential introduction of one or more heterologous glycosidase or glycosyltransferase activities. Each engineering step involves transformation of a methylotrophic yeast strain with an appropriate vector, cultivation of a number of transformants, assessment of N-glycans and heterologous protein expression, and selection of an efficient clone based on the analysis prior to initiating the next engineering step. The efficiency of one engineering step is believed to affect the efficiency of the subsequent engineering step. Thus, the sequential introduction of heterologous glycosidase or glycosyltransferase activities and selection of transformants at each engineering step are critical to obtaining glycoproteins having homogeneous glycoforms characterized by a predominant N-glycan structure.

As used herein, the engineering steps made to methylotrophic yeast, as depicted in FIG. 1a, are also referred to as “Glycoswitch” steps.

The vectors suitable for use in practicing the present invention and capable of introducing a heterologous glycosidase and glycosyltransferase activity are also referred to as “Glycoswitch vectors” or in abbreviation in FIG. 1a, “pGS”. Examples of Glycoswitch vector include pGS-M8, pGS-M5, pGS-GnT-I, pGS-GnT-1-HIS, pGS-GalT, pGS-ManII, and pGS-GnT-II, as described in Table 4.

The term “heterologous” is used herein to indicate that a molecule is placed in a genetic, molecular or cellular environment that is different than its native environment. For example, a methylotrophic yeast strain can be transformed with a nucleic acid coding for a heterologous protein, i.e., a protein which the native, non-engineered methylotrophic yeast strain does not produce, and which can be a desirable glycosylation enzyme or a glycoprotein to be produced as a product. The resulting engineered strain will express the heterologous protein.

The sequential modifications, the enzymes and vectors suitable for use in the modification, and the steps involved for each modification or engineering step, are now described in details. While Pichia pastoris is specifically discussed, the engineering strategy applies to other species of Pichia, including but not limited to Pichia methanolica, Pichia angusta (formerly Hansenula polymorpha), Pichia stipitis, and Pichia anomala as well as other methylotrophic yeasts. Methylotrophic yeasts are those capable of growth on methanol, and include yeasts of the genera Candida, Hansenula (such as H. polymorpha, now classified as Pichia angusta), Torulopsis, and Pichia. A particularly useful strain of Pichia pastoris is the well characterized strain GS115 (De Schutter et al., Nature Biotechnol 27:561, 2009).

The term “biosimilar” refers to a recombinant protein or group of recombinant proteins that are similar to an innovative protein drug product that, while having similar therapeutic or biological activity, differ from the innovative protein drug product in method of manufacture, structure (e.g., the difference being one or more amino acid substitutions, insertions or deletions), or post-translational modifications. A biosimilar may or may not be therapeutically substituted for the innovative product. Post-translational N-glycan variants of a therapeutic protein produced by the Pichia strains of the invention may vary from an innovative protein by being made in Pichia and having a nearly homogenous decoration of N-glycans instead of lacking N-glycan or having a heterogeneous decoration of N-glycans.

Subcellular Targeting of Enzymes

Since N-glycosylation is a sequential process, where one enzyme produces the substrate for the next, correct subcellular targeting of the introduced proteins is of critical importance. α-1,2-mannosidase, whose activity converts Man₈GlcNAc₂ (M8, FIG. 1a) N-glycans to Man₅GlcNAc₂ (M5, FIG. 1a) N-glycans, is targeted to the ER. In a preferred embodiment, this enzyme is targeted by fusing it C-terminally with the ER-retention or localization signal, HDEL (SEQ ID NO: 1) or KDEL (SEQ ID NO: 2), which labels soluble proteins for retrieval from the Golgi to the ER (FIG. 1b). Most of the glycosidases and glycosyltransferases catalyzing Golgi N-glycosylation reactions are type II membrane proteins. Their N-terminal region (cytosolic tail, transmembrane domain and part of the luminal ‘stem’ region) is responsible for correct subcellular localization. However, to eliminate the possibility that mammalian Golgi retention signals are not necessarily functional in yeast, in preferred embodiments, the N-terminal localization signal of each introduced glycosylation enzyme is replaced with the amino-terminus of a yeast protein with a known subcellular localization—i.e, the introduced enzymes are hybrid proteins with a yeast N-terminal localization domain (FIG. 1b). Examples of yeast Golgi localization signals suitable for use are provided in Table 4, including amino acids 1-100 of S. cerevisiae Kre2 protein, amino acids 1-36 and amino acids 1-46 of S. cerevisiae Mnn2 protein as well as others well known in the art.

Functional Part or Enzymatically Active Fragment

By “functional part” or “enzymatically active fragment” of a glycosylation enzyme is meant a polypeptide fragment of a glycosylation enzyme which substantially retains the enzymatic activity of the full-length protein. By “substantially” is meant at least about 40%, or preferably, at least 50%, 60%, 70%, 80%, 90% or more of the enzymatic activity of the full-length protein is retained. For example, as illustrated by the present invention, the catalytic domain of an α-1,2-mannosidase constitutes a “functional part” of the α-1,2-mannosidase. Those skilled in the art can readily identify and make functional parts of a glycosylation enzyme based on information available in the art and a combination of techniques known in the art. The activity of a particular polypeptide fragment of interest, expressed and purified from an appropriate expression system, can be also verified using in vitro or in vivo assays known in the art.

Disruption of the OCH1 α-1,6-mannosyltransferase Gene

As the Och1p α-1,6-mannosyltransferase initiates the “outer chain”, disruption of OCH1 is the first step in the engineering of the N-glycosylation pathway of P. pastoris.

According to the present invention, a disruption in the OCH1 gene can result in either the production of an inactive protein product or no product. The disruption may take the form of an insertion of a heterologous DNA sequence into the coding sequence and/or the deletion of some or all of the coding sequence, based on well-known techniques such as homologous recombination (Methods in Enzymology, Wu et al., eds., vol 101:202-211, 1983).

An OCH1 knock-out vector can be constructed to effect the disruption. Given that the disruption of the OCH1 gene results in the production of the M8 glycoform, such a vector is also referred to as a “pGS-M8” vector. The design of a pGS-M8 vector can depend on the type of homologous recombination desired.

In one embodiment, the pGS-M8 vector includes a selectable marker gene, which is flanked by portions of the OCH1 gene sequences of sufficient length to mediate double homologous recombination. A fragment of such vector, which contains the selectable marker gene flanked by OCH1 gene sequences, are then introduced by transformation into host methylotrophic yeast cells. Integration of the linear fragment into the genome and the disruption of the Och1 gene can be determined based on the selection marker and can be further verified by, for example, Southern Blot analysis.

In another, preferred embodiment, the pGS-M8 vector is constructed in such a way to achieve disruption after single homologous recombination. Such a vector includes a fragment of the OCH1 gene, which fragment is devoid of any promoter sequence and encodes none or an inactive fragment of the Och1 protein. By “an inactive fragment”, it is meant a fragment of the Och1 protein which has, preferably, less than about 10% and most preferably, about 0% of the activity of the full-length OCH1 protein. The OCH1 DNA fragment is placed in the vector in such a way that no known promoter sequence is operably linked to the OCH1 sequence, and optionally a stop codon and a transcription termination sequence are operably linked to the OCH1 fragment. This vector can be linearized at a site within the OCH1 sequence and transformed into a methylotrophic yeast strain using any of the methods known in the art. A single homologous recombination event will results in an OCH1 fragment under control of the OCH1 promoter that does not translate to a functional Och1 protein; and a second OCH1 copy that cannot be transcribed because of the absence of a promoter. Any translation of mRNA that would result from cryptic promoter activity is mitigated by the presence of the stop codon included in the construct.

A specific example of such pGS-8 vector is shown in FIG. 1c and also characterized in Table 4. About half of the antibiotic-resistant clones obtained upon transformation with this vector have been shown to have integration at the targeted site, which is much more than using classical double homologous-recombination knockout for this particular locus (where several hundred clones need to be screened).

The resulting M8 strain, i.e., the strain that produces Man₈GlcNAc₂ as the predominant N-glycan species, has a much reduced ability to modify glycoproteins (both endogenous and heterologous) with hyperglycosyl N-glycans. Because the M8 structure is the substrate for several endogenous glycosyltransferases besides Och1p, the N-glycan profile may not be entirely or 100% homogeneous. However, the heterogeneity of glycoproteins produced in M8 strains is strongly reduced (i.e. ‘smearing’ on SDS-PAGE due to hyperglycosylation is largely mitigated), especially after screening of transformants based on glycan analysis.

Introduction of ER-Localized α-1,2-mannosidase

The next step involves the introduction of a nucleotide sequence coding for an α-1,2-mannosidase or a functional fragment thereof into the methylotrophic yeast. The expressed enzyme removes all terminal α-1,2-linked mannose residues from Man₈GlcNAc₂ to produce Man₅GlcNAc₂ (see FIG. 1a).

The nucleotide sequence encoding an α-1,2-mannosidase or a functional fragment thereof can derive from any species as long as it converts Man₈GlcNAc₂ to produce the correct isomeric form of Man₅GlcNAc₂. A number of such α-1,2-mannosidase genes have been cloned and are available to those skilled in the art, including mammalian genes encoding, e.g., a murine α-1,2-mannosidase IA and IB (Herscovics et al. J. Biol. Chem. 269: 9864-9871, 1994; Lal et al. J. Biol. Chem. 269: 9872-9881, 1994), a human α-1,2-mannosidase (Tremblay et al. Glycobiology 8: 585-595, 1998), as well as fungal genes encoding, e.g., an Aspergillus α-1,2-mannosidase (msdS gene), a Trichoderma reesei α-1,2-mannosidase (Maras et al. J. Biotechnol. 77: 255-263, 2000). Protein sequence analysis has revealed a high degree of conservation among the eukaryotic α-1,2-mannosidases identified so far (Gonzalez et al., Mol. Biol. Evol. 17(2): 292-300, 2000).

Preferably, the nucleotide sequence for use in the present vectors encodes a fungal α-1,2-mannosidase or a functional fragment, more preferably, a Trichoderma reesei α-1,2-mannosidase, and more particularly, the catalytic domain of the Trichoderma reesei α-1,2-mannosidase described by Maras et al., J. Biotechnol. 77: 255-63 (2000).

The α-1,2-mannosidase or a functional fragment should be targeted to the ER. In a preferred embodiment, this enzyme is targeted by fusing it C-terminally with the ER-retention or localization signal, HDEL (SEQ ID NO: 1) or KDEL (SEQ ID NO: 2),

After transformation and proper screening, a selected M5 strain is obtained that modifies its glycoproteins predominantly with Man₅GlcNAc₂ structures. Since most endogenous glycosyltransferases are not able to act on this structure, the N-glycan profile of such a M5 strain is very homogeneous.

For easy conversion of any expression strain into a M5 strain, the α-1,2-mannosidase-HDEL can be inserted into the och1 inactivation vector. The resulting combination vector is designated pGlycoSwitchM5. A specific example of pGSM5 is characterized in Table 4.

Maturation of N-glycans into Hybrid- and Complex-Type Structures

The first step in the maturation of N-glycans into hybrid- and complex-type structures is the addition of a β-1,2-linked GlcNAc residue to the α-1,3-mannose of the trimannosyl core, a reaction catalyzed by GlcNAc transferase I (GnT-I). Introduction and expression of GnT-I or a functional fragment thereof can be achieve by the vector, pGlycoSwitchGnT-I.

According to the present invention, the nucleotide sequence encoding a GlcNAc-transferase I (GnT-I) for use in the present invention can derive from any species, e.g., rabbit, rat, human, plants, insects, nematodes and protozoa such as Leishmania tarentolae, or can be obtained through protein engineering experiments. Preferably, the nucleotide sequence encodes a human GnT-I.

The GnT-I or a functional part thereof is targeted to the Golgi apparatus of the recipient methylotrophic yeast. This can be achieved by including a yeast Golgi localization signal in the GnT-I protein or a functional part thereof. In a preferred embodiment, the catalytic domain of human GnT-I is fused to the N-terminal domain of S. cerevisiae Kre2p, a glycosyltransferase with a known cis/medial Golgi localization, resulting in the vector pGS-GnT-I. A specific example of such a vector is characterized in Table 4. The Kre2-GnT-I fusion construct is introduced by transformation of methylotrophic yeast with the vector pGS-GnT-I. Expression of the Kre2-GnT-I hybrid protein in a M5 strain results in a strain (GnM5) that modifies its glycoproteins with GlcNAcMan₅GlcNAc₂ N-glycans.

The next step in the maturation of N-glycans into hybrid- and complex-type structures is the addition of a galactose residue in β-1,4-linkage to the β-1,2-GlcNAc, a reaction catalyzed by β-1,4-galactosyltransferase 1, using UDP-Gal as donor substrate.

In one embodiment, this addition of a galactose residue is achieved by further introducing to a GnM5 strain with a pGlycoSwitchGalT vector. Such vector contains a nucleotide sequence coding for β-1,4-galactosyltransferase 1 or a functional part thereof. The GalT or a functional part thereof can be of an origin of any species, including human, plants (e.g. Arabidopsis thaliana), insects (e.g. Drosophila melanogaster). A preferred GalT for use in the present invention is human GalTI. The GalT or a functional part thereof is genetically engineered to contain a Golgi-retention signal and is targeted to the Golgi apparatus. A preferred Golgi-retention signal is composed of the first 100 amino acids of the Saccharomyces cerevisiae Kre2 protein.

In another embodiment, the pGSGalT vector drives the expression of a tripartite fusion protein composed of the catalytic domain of human β-1,4-galactosyltransferase 1, the entire Schizosaccharomyces pombe UDP-Gal 4-epimerase, and the Golgi-localization domain of S. cerevisiae Mnn2p. GalT adds a galactose residue in β-1,4-linkage to the β-1,2-GlcNAc, using UDP-Gal as donor substrate. The epimerase supplies the Golgi complex with sufficient amounts of UDP-Gal, by converting UDP-Glc into UDP-Gal. Examples of such a vector are provided in Table 4.

The resulting GalGnM5 strain modifies its glycoproteins with hybrid-type GalGlcNAcMan₅GlcNAc₂ structures.

Production of Mammalian Complex-Type Structures

The first step towards the production of mammalian complex-type structures is the introduction of mannosidase II (Man-II) activity. Man-II is responsible for the removal of both terminal α-1,3- and α-1,6-mannoses from GlcNAcMan₅GlcNAc₂ N-glycans. The presence of a terminal β-1,2-linked GlcNAc residue on the α-1,3-arm is essential for its activity.

Introduction of the Man-II activity can be achieved by transforming with a pGS-Man-II vector, which contains a nucleotide sequence coding for a Man-II protein or a functional fragment thereof. The Mannosidase II genes have been cloned from a number of species including mammalian species.

In a preferred embodiment, the catalytic domain of Drosophila melanogaster Man-II (GenBank Accession No. X76522, amino acids 75-1108) was fused to amino acids 1-36 of the Golgi-localization domain of S. cerevisiae Mnn2p, as exemplified in Table 4. Expression of this fusion protein in a GnM5 strain results in a GnM3 strain, the latter modifies its glycoproteins with GlcNAcMan₃GlcNAc₂ N-glycans. Expression of the Mnn2DmMan-II fusion protein in a GalGnM5 strain results in a strain that modifies its glycoproteins with GalGlcNAcMan₃GlcNAc₂ structures (GalGnM3).

Introduction of Man-II is a difficult step in the engineering process, as it may significantly influencing the growth characteristics (Box 1), and results in a heterogeneous N-glycan profile. Since the products of Man-II appear to be (non-natural) substrates for endogenous Pichia glycosyltransferases implicated in outer chain synthesis, this growth problem can be largely solved by introduction of GnT-II, which competes with these endogenous mannosyltransferases for the same substrate (i.e. product of ManII).

The final step towards the production of biantennary complex-type N-glycans with terminal galactose is the introduction of a GlcNAc transferase II (GnT-II) activity. This enzyme catalyzes the addition of a second β-1,2-linked GlcNAc residue to the free α-1,6-mannose of the trimannosyl core.

Introduction of the GnT-II activity can be achieved by transforming with a pGS-GnT-II vector, which contains a nucleotide sequence coding for a GnT-II protein or a functional fragment thereof. GnT-II genes have been cloned from a number of species including mammalian species and can be used in the present invention.

In a preferred embodiment, the catalytic domain of rat GnT-II (GenBank Accession No. U21662) to the N-terminal part (amino acids 1-36) of S. cerevisiae Mnn2p. Transformation of a GnM3 strain with the vector pGlycoSwitchGnT-II results in a strain that modifies its glycoproteins with GlcNAc₂Man₃GlcNAc₂ structures, termed Gn2M3. Similarly, transformation of a GalGnM3 strain results into a Gal2Gn2M3 strain, i.e. a strain that can synthesize Gal₂GlcNAc₂Man₃GlcNAc₂ N-glycans.

Introduction of a Glycoprotein of Interest

The present glycoengineering strategy permits the production of a glycoprotein of interest having a particular N-glycan structure as the predominant glycoform, or a series of the glycoprotein, each having a different, predominant glycoform. One can generate a series of methylotrophic yeast strains, each producing one of the glycoform as depicted in FIG. 1a as the predominant glycoform, then introduce a nucleotide sequence encoding the glycoprotein of interest to each of the glycoengineered strains. Alternatively and preferably, one can start with a methylotrophic yeast strain already optimized to express the glycoprotein of interest. For example, one can start from a GS115 wild type P. pastoris strain transformed with a pPIC9-derived target protein expression plasmid to complement its histidine auxotrophy. Linearization of the expression vector in the HIS4 gene directs integration of the expression vector to the HIS4 gene locus in the genome. Since considerable clonal variation in expression levels occurs in P. pastoris, at least 10, 15, 20, 25 or more transformants should be evaluated in small-scale expression experiments to identify a clone that expresses the protein at a high level.

Glycoengineering Protocol

Modification of the yeast glycosylation pattern is achieved by the disruption of an endogenous glycosyltransferase gene (OCH1) and the step-wise introduction of one or more heterologous glycosidase and glycosyltransferase activities as depicted in FIG. 1a. Each engineering step consists of a number of stages: transformation with an appropriate Glycoswitch vector, cultivation of a number of transformants, sugar analysis and heterologous protein expression analysis, and selection of a desirable clone.

Transformation of a methylotrophic yeast strain can be achieved using various methods known in the art, including the spheroplast technique (Cregg et al. 1985), the whole-cell lithium chloride yeast transformation system (EP 312,934), electroporation and PEG1000 whole cell transformation procedures (see, e.g., Cregg and Russel Methods in Molecular Biology: Pichia Protocols, Chapter 3, Humana Press, Totowa, N.J., pp. 27-39 (1998)). A preferred transformation method is electroporation, which results in high transformation efficiencies and does not involve digestion of the cell wall, of which mannoproteins form an important part.

Transformed yeast cells are preferably plated onto solid media and can be selected by using appropriate techniques including but not limited to culturing auxotrophic cells after transformation in the absence of the biochemical product required, culturing in the presence of an antibiotic, among others, depending on the selectable marker gene contained in the vector being introduced. Selectable marker genes include those which either complement host cell auxotrophy, such as URA3, LEU2 and HIS3 genes, or provide resistance to an antibiotic. Preferred choices of antibiotics are listed in Table 2. Other suitable selectable markers include the CAT gene, which confers chloramphenicol resistance on yeast cells, or the lacZ gene, which results in blue colonies on indicator plates due to the expression of active β-galactosidase. Transformants can also verified by e.g., Southern Blot or PCR analysis, to confirm integration of the expression cassette into the genome.

After colonies appear on solid media, a semi-high throughput small-scale expression protocol that allows for the simultaneous analysis of a number of clones or colonies. In accordance with the present invention, at least 15, 20, 25, 30, or even 35 clones are included in the small scale liquid cultivation. By “small scale” cultivation, it is meant cultivation in liquid media of not more than 100 ml, or preferably not more than 75 ml, or even more preferably, about 50 ml or less, to even less than 5 ml, or less than 2 ml, or even 1 ml. The purpose of the small-scale cultivation step is (1) to identify clones that have the desired N-glycosylation profile and (2) to evaluate the impact (if any) of this particular glycoengineering step on the production level of the protein of interest. The exact specifications of the applied small-scale production protocol depend on the strain that is being glyco-engineered. The protocol should be adapted to the protein of interest and to the promoter used (e.g. AOX1, GAP, among others) that drives the expression of the gene of interest. Two example small-scale production protocols are described in Example 1 below.

N-glycan analysis is conducted with each of clones having cultivated in a small-scale liquid medium. If the clone also expresses a heterologous glycoprotein of interest, protein expression analysis of the heterologous protein will also be conducted.

To perform N-glycan analysis, one can analyze the glycoproteins present in the growth medium. Alternatively, one can analyze the endogenous mannoproteins of the yeast's cell wall. A simple, high throughput protocol is provided herein (Steps 39-50 in Example 1, which results in a crude cell wall extract containing a mixture of mannoproteins and β-glucan. Loading of the crude cell wall extracts on PVDF membranes results in binding of the mannoproteins to the membrane. Since the PVDF membranes do not bind glucans, contaminating cell wall β-glucans can be easily removed (and prevented from interfering with the subsequent N-glycan profiling). The protein-bound N-glycans are subsequently analyzed by, e.g., DNA sequencer assisted (DSA), fluorophore assisted carbohydrate electrophoresis (FACE) (see Example 2), or MALDI-TOF MS.

According to the present invention, DSA-FACE is a preferred approach of performing N-glycan analysis. Essentially, N-glycans are released from glycoproteins of a selected source by treatment with peptide: N-glycosidase F (PNGase F). Subsequently, the released N-glycans are derivatized with the fluorophore 8-aminopyrene-1,3,6-trisulfonate (APTS) by reductive amination. After removal of excess APTS, the labeled N-glycans are analyzed with an ABI 3130 DNA sequencer.

Heterologous protein expression analysis can be performed by SDS-PAGE, Western blotting, and/or ELISA, depending on the protein.

Based on the N-glycan and protein expression analysis data, one clone is selected that can be subject to a further glycoengineering round (i.e., introducing the next glycosylation enzyme in line as depicted in FIG. 1a). This procedure can be repeated until the desired glyco-strain has been created (FIG. 1d).

The procedure depicted in FIG. 1a results ultimately in the modification of glycoproteins with complex type N-glycans, with the Gal₂GlcNAc₂Man₃GlcNAc₂ structure (FIG. 1a, indicated as Gal2Gn2M3) as the final product. One can further modify this structure, for example by sialylation. For example, in vivo engineering for the sialylation of glycoproteins in Pichia has been reported (Hamilton et al., Science 313, 1441-1443 (2006)) which involves copying the entire CMP-N-acetylneuraminic acid (CMP-NANA) synthesis pathway, a CMP-NANA transporter and a sialyltransferase into the yeast system. Additionally, one can make further modifications to the engineered yeast to reduce or eliminate undesirable of non-human type O-glycosylation in methylotrophic yeasts such as Pichia. Inhibition of O-glycosylation with small-molecule drugs has also been reported (Kuroda et al., Appl. Environ. Microbiol. 74, 446-453 (2008)) and can be applied as well.

Selection of Engineered Pichia

The goal of the system of the invention is to identify candidate engineered Pichia strains that produce variously glycosylated forms of a heterologous protein and select a useful form(s) of the protein. The selected protein may be a biosimilar therapeutic protein. In order to select the useful form(s) of the heterologous protein, selection criteria are specified. Since proteins may have different activities that are considered useful, the criteria may vary depending on the intended use. For example, heterologous proteins made in Pichia may be useful as industrial enzymes, animal nutrition, and pharmaceuticals among other uses. A preferred use is therapeutic proteins for pharmaceutical use.

Proteins used as pharmaceuticals have a number of properties that may vary with the type of N-glycans carried by the protein. These properties include the physical properties of the pharmaceutical substance that can affect storage, formulation properties that can affect its ability to be effectively delivered by various routes of administration, and biological properties that affect the pharmacodynamics and pharmacokinetics of the therapeutic protein, as well as its toxicity to the patient. For example, the type of N-glycans present on a protein can affect its ability to be lyophilized, its tendency to aggregate in solution or in storage, its degradation rate in storage, its ability to be formulated for administration, whether by oral, parenteral or topical administration, its rate of absorption, distribution, metabolism and elimination from the patient, its ability to interact with its cognate receptor and its ability to elicit immunogenic or other undesirable responses in the patient. Important properties for the therapeutic protein of interest can be determined and criteria established for selecting a useful heterologous protein based on those criteria.

Depending on the criteria established, important properties of the therapeutic proteins with the varied N-glycans could include the affinity or avidity of the protein for its receptor, the enzymatic activity of the protein, its solubility, its tendency to aggregate in solution or lyophilized form, its stability in storage, its distribution in the body once administered, its half-life in the body, its route of elimination, its ability to elicit neutralizing antibodies or other unwanted reactions in the patient. Assays for such properties are well known in the pharmaceutical arts and can be set up to measure the variance of these properties among the various candidate glycosylated proteins.

The usefulness of this system for selecting a useful therapeutic protein from a group of proteins that vary only in their N-glycan decoration is important for selecting biosimilar therapeutic proteins. Biosimilars can be, for example, protein therapeutics that vary from a reference therapeutic protein in their structure, method of manufacture, or post-translational modifications, but exhibit therapeutic activity similar to the reference protein. In some cases the biosimilar may be therapeutically substituted for the reference pharmaceutical. The Pichia N-glycan system disclosed herein is useful for developing a series of candidate biosimilar recombinant proteins.

The present invention is further illustrated but by no means limited by the following examples.

### Example 1

### Reagents

Reagents used in Example 2 included antibiotics, such as Blasticidin S (Fluka), Zeocin (Invitrogen), Nourseothricin (Werner BioAgents), Geneticin G418 (Invitrogen), and Hygromycin B (Calbiochem); Bacto Agar (Difco), Bacto peptone (Difco), Bacto yeast extract (Difco), Biotin (Sigma), BMGY (see REAGENT SETUP), BMMY (see REAGENT SETUP), Citric acid (Calbiochem), Deionized water (dd-water), DTT (Sigma), Glucose monohydrate (Merck), Glycerol (Biosolve), HEPES (Sigma), Methanol (Biosolve), NaCl (Merck); restriction enzymes, such as AvrII (New England Biolabs), BsiWI (New England Biolabs), BstBI (New England Biolabs), PmeI (New England Biolabs), SapI (New England Biolabs), Sorbitol (Sigma), YNB (yeast nitrogen base) without amino acids (Difco) and YPD media and plates (see REAGENT SETUP).

TABLE 2
Antibiotics

| Antibiotic | Final concentration (μg/ml) |
|---|---|
| Blasticidin | 500 |
| Zeocin | 100 |
| Nourseothricin | 100 |
| G418 | 350 |
| Hygromycin B | 150 |

Equipment

Equipments used in Example 2 included 24-well culture plates (X25 UNIPLATE 10000 24 PP; Whatman), AIRPORE tape (Qiagen), autoclave, baffled shake flask (1 liter), centrifuge (Sorvall), CryoTube vials (1 ml (Nunc)), Eppendorf® Safe-Lock® microcentrifuge tubes, electroporation cuvettes, 2 mm (BioRad), FALCON 50 ml conical tubes (BD), GENE PULSER electroporator (BioRad), incubator shaker at 30° C., microcentrifuge tube closures (Sherlock® tube closures; USA Scientific), Nucleospin® Extract-II kit (Macherey-Nagel), oven at 37° C., oven at 30° C., Safe-Lock eppendorf tubes (2 ml (Eppendorf)), spectrophotometer (able to measure 600 nm), SpeedVac vacuum dryer (Savant), tabletop centrifuge, and vortex.

Reagent Setup

1 M potassium phosphate buffer pH 6.0—Dissolve 23 g K₂HPO₄ and 118 g KH₂PO₄ in 1000 ml deionized water and confirm that the pH=6.0±0.1 (if the pH needs to be adjusted, use phosphoric acid or KOH). Autoclave for 20 minutes at 121° C. The shelf life for this solution is >1 year.

13.4% YNB w/o aa—Dissolve 67 g YNB (yeast nitrogen base) without amino acids in deionized water to a final volume of 500 ml. Heat the solution to dissolve the YNB completely. Filter sterilize. The shelf life for this solution is >1 year when stored at 4° C.

500× biotin—Dissolve 20 mg biotin in 100 ml deionized water and filter sterilize. Stored at 4° C. the shelf life for this solution is 1 year.

BMGY medium—1% (wt/vol) yeast extract, 2% peptone (wt/vol), 1.34% (wt/vol) YNB w/o amino acids, 100 mM potassium phosphate buffer (pH 6.0), 4×10⁻⁵% (wt/vol) biotin, and 1% glycerol (vol/vol). For 500 ml, dissolve 5 g yeast extract and 10 g peptone in 400 ml deionized water and autoclave for 20 minutes at 121° C. Cool to room temperature (21° C.) and add 50 ml sterile 1 M potassium phosphate buffer (pH 6.0), 50 ml sterile 13.4% YNB w/o amino acids, 1 ml 500× biotin, and 5 ml sterile 100% glycerol. The shelf life for this solution is approximately 2 months.

BMMY medium—1% (wt/vol) yeast extract, 2% peptone, 1.34% (wt/vol) YNB w/o amino acids, 100 mM potassium phosphate buffer (pH 6.0), 4×10⁻⁵% (wt/vol) biotin, and 1% (vol/vol) methanol. For 500 ml, dissolve 5 g yeast extract and 10 g peptone in 400 ml deionized water and autoclave for 20 minutes at 121° C. Cool to room temperature and add 50 ml sterile 1 M potassium phosphate buffer (pH 6.0), 50 ml sterile 13.4% YNB w/o aa, 1 ml 500× biotin, and 5 ml 100% methanol. The shelf life for this solution is approximately 1 month.

YPD medium—1% (wt/vol) yeast extract, 2% (wt/vol) peptone, and 2% (wt/vol) glucose monohydrate. Dissolve 5 g yeast extract, 10 g peptone, and 10 g glucose monohydrate in deionized water to a final volume of 500 ml. Autoclave for 20 minutes at 121° C. The shelf life for this solution is approximately 1 month.

YPD plates—1% (wt/vol) yeast extract, 2% (wt/vol) peptone, 2% (wt/vol) glucose monohydrate, and 1.5% (wt/vol) agar. Dissolve 5 g yeast extract, 10 g peptone, 10 g glucose monohydrate and 7.5 g agar in deionized water to a final volume of 500 ml. Autoclave for 20 minutes at 121° C. Cool the mixture while stirring until below 50° C., add the appropriate antibiotics (Table 2), and pour plates. Store plates at 4° C. for no longer than one month.

Plasmid DNA Preparation

All GlycoSwitch vectors and vectors for overexpression of mIL-10, mGM-CSF and mIL-22 were prepared from cultures of E. coli MC1061 using standard plasmid purification kits which yielded sequencing-grade, low-salt preparations of the plasmids (such as those that are obtained from Qiagen or Machery-Nagel). The plasmid concentration was estimated through absorbance measurements at 260 nm.

Procedure

Pichia Transformation: Vector Linearization
1| Digest 5-10 μg of plasmid DNA with the appropriate restriction enzyme (Table 4). Use enzyme/substrate ratios recommended by the manufacturer. Complete linearization is best ascertained through analyzing an aliquot of the restriction digest mixture on an agarose gel.
2| The mixture is subsequently desalted, which can be done using Macherey-Nagels Nucleospin® Extract-II kit. Contaminants like salts and enzymes are removed by a simple washing step. Pure DNA is finally eluted under low ionic strength conditions. It is critical that the DNA mixture is “salt-free” as salt causes arching during the electroporation step.
3| Finally, the mixture is evaporated to dryness (SpeedVac, this typically takes about 30 min without heating) and the DNA is resuspended in 10 μl of ultrapure water.

Pichia Transformation: Preparation of Competent Pichia Cells
4| Transfer 10 ml sterile YPD medium (see REAGENT SETUP) to a 50 ml falcon tube. Work aseptically throughout this procedure.
5| Inoculate the tube with one colony from the P. pastoris clone of interest and grow overnight at 250 r.p.m. and 30° C.
6| Next day, measure the OD₆₀₀ of the overnight pre-culture (an absorbance of 1 in a 1 cm cuvette at 600 nm is about 2×10⁷ cells per ml).
7| Transfer 250 ml sterile YPD medium to a 1 l baffled shake flask.
8| Inoculate the shake flask with X ml of the pre-culture and grow overnight to an OD₆₀₀ of 1.3-1.5. X can be calculated from the following formula: X×OD×2^{y}=250×1.4; where OD is the OD₆₀₀ of the pre-culture, y is the number of generations the culture will be grown, 250 is the volume of the culture (in ml) and 1.4 is the desired OD₆₀₀ value.
   The GS115 wild type P. pastoris strain has a generation time of approximately 2 hours. However, some glycoengineering steps result in an increase doubling time of the resultant strain (discussed in more detail in Table 3).
9| When the shake flask culture has reached the desired OD₆₀₀ value, centrifuge the cells at 1,500 g for 5 minutes at 4° C.
10| Resuspend the cell pellet in 100 ml YPD, 20 ml HEPES buffer pH 8 to which 2.5 ml 1M DTT has been added. 1M DTT should be prepared freshly. Transfer the mixture back the 1 l baffled flask and shake for 15 minutes at 30° C.
11| Add 125 ml ultrapure water and centrifuge at 1,500 g for 5 minutes at 4° C. Keep the cells on ice during all subsequent manipulations.
12| Resuspend the cell pellet in 250 ml of ice-cold, sterile ultrapure water. Centrifuge the cells at 1,500 g for 5 minutes at 4° C.
13| Resuspend the cell pellet in 125 ml of ice-cold, sterile ultrapure water. Centrifuge the cells at 1,500 g for 5 minutes at 4° C.
14| Resuspend the cell pellet in 20 ml of sterile, ice-cold 1 M sorbitol. Centrifuge the cells at 1,500 g for 5 minutes at 4° C.
15| Resuspend the cells in 500 μl of sterile, ice-cold 1 M sorbitol. The cells are now electrocompetent and should be used as soon as possible. The purpose of all these washing steps is to ensure that the cells are “salt-free” (as salt causes arcing during the electroporation step) while suspending them in an osmotically stabilizing solution.

Pichia Transformation: Transformation
16| Mix 80 μl of the competent cells from step 15 with the linearized DNA from step 3 and transfer them to an ice-cold 0.2 cm electroporation cuvette. Equilibrate on ice for 5 minutes.
17| Pulse the cells according to the parameters suggested for yeast by the manufacturer of the electroporation instrument being used.
18| Immediately add 1 ml of ice-cold 1 M sorbitol.
19| Transfer the cells to a sterile 15 ml tube and incubate without shaking at 30° C. for 1 to 2 hours.
20| Plate 10, 50 and 200 μl of this cell suspension on YPD plates containing the appropriate antibiotic.
21| Incubate for 2 to 3 days at 30° C. until colonies appear.
22| Isolate >20 single clones by picking individual colonies and streaking them on YPD plates containing the appropriate antibiotic.

Small-Scale Cultivation
23| Small-scale cultivation can either be done by the 50-ml falcon tube method (Option A) or the 24-well plate method (Option B).

(A) 50-ml Falcon Tube Method
i. Grow each isolated single clone from step 22 in a 50 ml falcon tube containing 10 ml BMGY medium at 30° C. while shaking (250 r.p.m.).
ii. After 48 hours of growth centrifuge the cultures at 3000 g for 5 minutes.
iii. Resuspend the cell pellets in 10 ml BMMY medium.
iv. To maintain induction, spike the cultures every 12 hours with 100 μl 100% methanol (1% final concentration). In this way, cultivate for another 48 hours at 30° C. (or shorter or longer, depending on the pre-determined optimum for your specific protein).
v. After the desired induction time, measure the OD600 and harvest the cultures by centrifugation (3000 g for 10 minutes).
vi. Freeze the supernatant at −20° C. until further use.
vii. Freeze the cell pellets at −20° C. until further use.

(B) 24-Well Plate Method
i. Aseptically inoculate each isolated single clone from step 22 in one well containing 2 ml BMGY medium.
ii. Seal the plate with Airpore tape.
iii. Incubate at 30° C., while shaking (250 r.p.m.)
iv. After 48 hours of growth centrifuge at 3000 g for 10 minutes.
v. Resuspend the cell pellets in 10 ml BMMY medium.
vi. To maintain induction, spike the cultures every 12 hours with 50 μl 100% methanol (1% final concentration). Using this method, cultivate for another 48 hours at 30° C. (or shorter or longer, depending on the pre-determined optimum for your specific protein).
vii. At the end of the induction phase, measure the OD600 and harvest the cultures by centrifugation (3000 g for 10 minutes).
viii. Freeze the supernatant at −20° C. until further use.
ix. Freeze the cell pellets at −20° C. until further use.

Cell Wall Mannoprotein Extraction
24| Wash the cells from Step 23A(vii) or 23B(ix) once with 1 ml of 0.9% NaCl in a 2 ml Safe-Lock eppendorf tube (2800 g, 1 minute).
25| Wash the cells once with 1 ml of dd-water (2800 g, 1 minute).
26| Resuspend the pellet in 1.5 ml of 20 mM Na-citrate buffer, pH 7.0.
27| Autoclave at 125° C. for 90 minutes. To avoid opening of the tubes, they should be locked with microcentrifuge tube closures. We use Eppendorf® SafeLock® microcentrifuge tubes because they do not melt/deform when subjected to Step 42.
28| When the samples are at room temperature, resuspend the cellular debris by vortexing.
29| Centrifuge at >13500 g for 5 minutes.
30| Collect the supernatant in a 15 ml tube and add 4 volumes (−6 ml) ice-cold methanol to precipitate the mannoproteins.
31| Stir the mixture overnight at 4° C., preferably by placing the samples on a rotating wheel.
32| Pellet the mannoproteins by centrifugation (3220 g, 4° C., 15 minutes).
33| Remove the supernatant fraction and wash the pellet with 0.5 ml methanol (3220 g, 4° C., 15 minutes).
34| Dry the pellet at 37° C. for 1 hour.
35| Dissolve the pellet in 50-100 μl dd-water.

N-Glycan Analysis
36| Perform N-glycan analysis of the samples from step 23 A vi, 23 B viii or 35 by DSA-FACE as described by Laroy et al. (Nat Protoc. 1: 397-405 (2006)) (reagent/equipment needs summarized in Example 2). If the protein of interest is secreted in the growth medium use 500-1000 μl of the supernatant fraction from step 23A(vi) or 23B(viii). In case of mannoprotein N-glycan analysis, use 50-100 μl from Step 35.

Protein Expression Analysis
37| To evaluate whether the performed glycoengineering step has affected the expression level of the protein of interest perform either SDS-PAGE, Western blot and/or ELISA. In order to be able to draw well-founded conclusions, differences in OD₆₀₀ at the end of the induction phase should be taken into account.

Clone Preservation
38| After having identified the best clone in terms of N-glycan profile and heterologous protein expression level grow it overnight in 5 ml YPD medium containing the appropriate antibiotics.
39| Make 1 ml aliquots containing 20-30% glycerol and store at −80° C.

Strain Start-Up from Preserved Clones Stored at −80° C.
40| Upon thawing, plate on YPD plates containing all appropriate antibiotics.
41| Isolate 5-10 single clones.
42| Grow these clones according to the 50-ml falcon tube method (Steps 23A) or the 24-well plate method (Steps 23B).
43| Perform N-glycan analysis according to Step 36.
44| Evaluate the protein expression level according to Step 37.
45| Based on the results from Steps 43 and 44 choose one clone to work further with.

Timing
Transformation (Steps 1-20): 3 days (hands-on time: 4 h).
Time needed for colonies to appear on plates (Step 21): 2-3 days (hands-on time: 0 h).
Isolation of single clones (Step 22): 2 days (hands-on time: 30 minutes).
Small-scale cultivation and cell wall/secreted protein preparation (Step 23-35): 4 days (hands-on time: 2-3 h).
N-glycan analysis by DSA-FACE (Step 36): 3-4 days (hands-on time: 1 day).
Protein analysis (Step 37): 1-2 days (during N-glycan analysis).
Total: 3 weeks/engineering step.

The slower doubling time of some glycoengineered strains (Table 4) does not negatively impact the timeline for introducing GlycoSwitch vectors, as the yeast growth steps in the protocol are not the bottlenecks.

Additional Considerations

When introducing the glyco-engineering constructs through single homologous recombination, a direct repeat is created at the genomic level. While homologous in- and out-recombination is a rare event in Pichia under normal cultivation conditions, stresses on the cells like electroporation and prolonged storage on agar plates at 4° C. could possibly induce out-recombination. Although loss of previously introduced constructs through out-recombination was sometimes observed upon introduction of the next GlycoSwitch vector in line, fully engineered clones were always identified. A more stringent selection for “good” transformants can be obtained by plating the electroporation mixture on YPD plates containing all previously used antibiotics.

When storing a glycoengineered strain on plate for longer than two weeks the appropriate antibiotics should be included.

Addition of antibiotics to the culture medium of both small-scale and large-scale protein expression cultivations is not necessary.

The simultaneous use of multiple antibiotics does not have any detrimental effects on the viability of the glycoengineered strains.

TABLE 3
Growth Characteristics of Glycoengineered Strains

| | | | Doubling | Maximum |
|---|---|---|---|---|
| | Introduced | % | time | growth rate μ |
| Strain | enzyme | conversion | (hours) | (h⁻¹) |
| GS115 (his4) | None | NA | 2.40 | 0.0048 |
| GS115mlL-10 | None | NA | 2.36 | 0.0049 |
| M5mlL-10 | Man-I | ND^{a} | 2.31 | 0.0050 |
| GnM5mlL-10 | GnT-I | 89.5% | 2.51 | 0.0046 |
| GalGnM5mlL-10 | GalT | 84.5% | 2.82 | 0.0041 |
| GalGnM3mlL-10 | Man-II | 90.8% | 3.61 | 0.0032 |
| Gal2Gn2M3mlL-10 | GnT-II | 95.5% | 4.62 | 0.0025 |

NA: not applicable; ND: not determined

TABLE 4
GlycoSwitch vectors

| | | | | | | | Linearization | |
|---|---|---|---|---|---|---|---|---|
| | | | | | | Integration | (Restriction | |
| Vector | Short name | Glycosyltransferase | Localization signal | Promoter | Selection | locus | enzyme) | Ref |
| pGlycoSwitchM8 | pGS-M8 | Δoch1 | NA | NA | Zeocin | OCH1 | BstBI | [9] |
| pGlycoSwitchM5 | pGS-M5 | Δoch1 | NA | NA | Blasticidin | OCH1 | BstBI | [9] |
| | | Man-I; T. reesei; | C-terminal HDEL tag | GAP | | | | |
| | | AA 25-523 | | | | | | |
| pGlycoSwitchGnT-I | pGS-GnT-I | GnT-I; H. sapiens; | Kre2p; S. cerevisiae | GAP | Zeocin | GAP | AvrII | [9] |
| | | AA 103-445 | AA 1-100 | | | | | |
| ^{a}pGlycoSwitchGnT-I-HIS | pGS-GnT-I- | GnT-I; H. sapiens; | Kre2p; S. cerevisiae | GAP | Histidine | GAP | DraIII | [9] |
| | HIS | AA 103-445 | AA 1-100 | | | | | |
| pGlycoSwitchGalT/1 | pGS-GalT | GalT; H. sapiens; | Mnn2p; S. cerevisiae | GAP | Nourseothricin | GAP | AvrII | [12] |
| | | AA 44-398 | AA 1-46 | | | | | |
| | | UDP-Gal | | | | | | |
| | | 4-epimerase; | | | | | | |
| | | S. pombe; | | | | | | |
| | | full length | | | | | | |
| pGlycoSwitchGalT/2 | pGS-GalT | GalT; H. sapiens; | Mnn2p; S. cerevisiae | AOX1 | Nourseothricin | AOX1 | PmeI | [12] |
| | | AA 44-398 | AA 1-46 | | | | | |
| | | UDP-Gal | | | | | | |
| | | 4-epimerase; | | | | | | |
| | | S. pombe; | | | | | | |
| | | full length | | | | | | |
| pGlycoSwitchMan-II/1 | pGS-Man-II | Man-II; | Mnn2p; S. cerevisiae | GAP | G418 | GAP | AvrII | [12] |
| | | D. melanogaster, | AA 1-36 | | | | | |
| | | AA 74-1108 | | | | | | |
| pGlycoSwitchMan-II/2 | pGS-Man-II | Man-II; | Mnn2p; S. cerevisiae | AOX1 | Hygromycin B | AOX1 | PmeI | [12] |
| | | D. melanogaster, | AA 1-36 | | | | | |
| | | AA 74-1108 | | | | | | |
| pGlycoSwitchGnT-II | pGS-GnT-II | GnT-II; | Mnn2p; S. cerevisiae | GAP | Hygromycin B | GAP | SapI | [12] |
| | | R. norvegicus; | AA 1-36 | | | AOX1 | BsiWI | |
| | | AA 88-443 | | | | | | |

NA: not applicable; AA amino acid
^{a}Allows zeocin-based selection for multiple copy integration of the recombinant protein-construct.

### Example 2

### DSA-FACE N-Glycan Profiling

To prepare samples for Fluorophore Assisted Carbohydrate Electrophoresis on capillary DNA-sequencers, N-glycans are released from the glycoproteins by treatment with peptide: N-glycosidase F (PNGase F). Subsequently, the released N-glycans are derivatized with the fluorophore 8-aminopyrene-1,3,6-trisulfonate (APTS) by reductive amination. After removal of excess APTS, the labeled N-glycans are analyzed with an ABI 3130 DNA sequencer. N-glycans of bovine RNase B and a maltodextrose ladder are included as references.

DSA-FACE Reagents
ABI 310 running buffer or ABI 3130 running buffer (Applied Biosystems)
Ammonium acetate (Merck)
APTS (Molecular Probes)
Citric acid (Calbiochem)
DMSO (Aldrich)
DTT (Sigma)
EDTA, dihydrate (Vel)
Exoglycosidases
   Jack Bean α-mannosidase (Sigma)
   Trichoderma reesei α-1,2-mannosidase (expressed and purified in our laboratory and available upon request)
   β-N-Acetylhexosaminidase (Prozyme)
   β-galactosidase (Prozyme)
GeneScan-500 LIZ size standard (when using ABI3130) or GeneScan-500 ROX size standard (when using ABI310) (Applied Biosystems)
HCl (Merck)
Iodoacetic acid (Sigma)
Malto-dextrin ladder, APTS-labeled (prepared in the author's laboratory and available upon request)
Methanol (Biosolve)
NaCl (Merck)
NaCNBH₃ (Acros) CAUTION: only small quantities are used, but this reagent is hazardous: vapors (HCN) formed in the acidic medium used for N-glycan derivatisation. Use in a well-ventilated hood.
PNGase F (New England Biolabs)
Polyvinylpyrrolidone 360 (Sigma)
POP-6 (when using ABI310) or POP-7 (when using ABI3130) Performance
Optimized Polymer (Applied Biosystems)
RNase B N-glycans, APTS-labeled (prepared in the author's laboratory and available upon request).
Sephadex G10 (Amersham)
Sodium acetate (Sigma)
Tris (Invitrogen)
Urea (Merck)

DSA-FACE Equipment
Adhesive tape for 96-well plates (Millipore)
Autoclave
Capillary (36 cm ABI 310) or capillary array (36 cm for ABI 3130; Applied Biosystems)
Centrifuge (Eppendorf 5810R)
Freezer at −20° C.
Genescan (ABI 310) or GeneMapper (ABI 3130) software (Applied Biosystems)
Genetic analyzer (ABI 310 or ABI 3130; Applied Biosystems)

Incubation oven at 37° C. and 50° C.
Microcentrifuge (Eppendorf 5417C)

Micropipettes (1000 μl, 200 μl, 50 μl, 10 μl and 2.5 μl)
Multiscreen-ImmobilonP (Millipore)
Multiscreen-Durapore 96-well filtration plates (Millipore)
Multiscreen column loader system, 100 μl (Millipore)
PCR thermocycler
PCR tubes
Reaction plates, 96-well (Applied Biosystems)
Refrigerator at 4° C.
Vacuum manifold for filtration plates (Millipore)
Vortex
Water bath

### Example 3

### Results

The workflow presented in FIGS. 1a and 1d allows engineering of the N-glycosylation pathway of any wild type P. pastoris strain. The construction of a strain that modifies its glycoproteins with Gal₂GlcNAc₂Man₃GlcNAc₂ N-glycans requires the consecutive integration of five GlycoSwitch vectors into the Pichia genome. Each of these plasmids contains a different dominant antibiotic resistance marker for selection. As a consequence, it is critical that the starting strain is still sensitive to all five antibiotics: blasticidin, zeocin, hygromycin, geneticin and nourseothricin. Some other combinations of engineering enzyme—selection marker are available (see Table 4), but not all. One can start from the GS115 wild type strain because its histidine auxotrophy provides an additional selection maker that allows selection for integration of a pPIC9-derived vector that drives the production of a protein of interest.

This Example describes results of three mouse proteins produced by the full engineering procedure described in Examples 1-2, with proteins spanning almost 2 orders of magnitude difference in expression level.

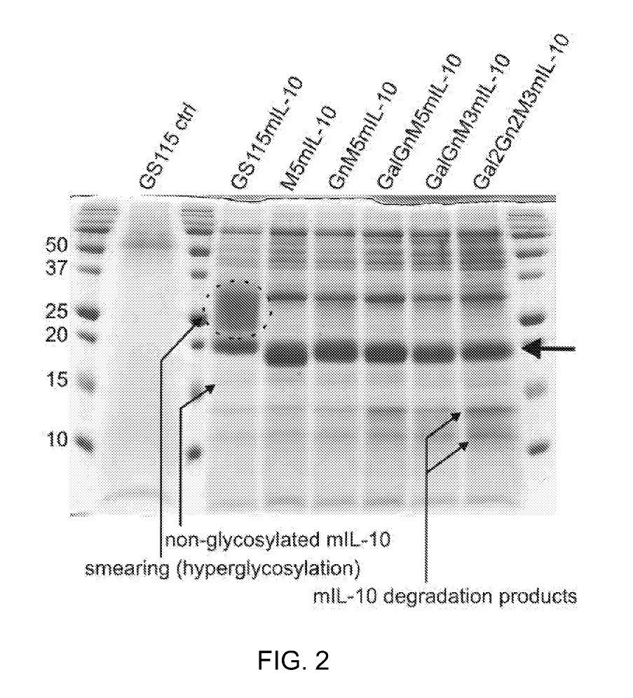

Mouse interleukin 10 (IL-10) is a ˜18.5 kDa homodimeric glycoprotein that is modified by the addition of one N-glycan structure near the N-terminus. Pichia-produced mIL-10 appears as a heavily smeared band on SDS-PAGE (FIG. 2). Removal of all N-glycans by treatment with PNGase F showed that this smearing was due to hyper-N-glycosylation. Production levels in P. pastoris are relatively low: 5-10 mg/l.

Mouse granulocyte-macrophage colony-stimulating factor (mGM-CSF) is a 124 amino acid monomeric glycoprotein with a predicted molecular weight of ˜14 kDa. It has two potential N-glycosylation sites and both sequons can be modified by the Pichia N-glycosylation machinery. Production levels in P. pastoris have been reported to be in the range of 200 mg/l (Sainathan et al., Protein Expr. Purif. 44: 94-103 (2005)).

Mouse interleukin 22 (mIL-22) is a 146 amino acid glycoprotein with a theoretical molecular weight of 16.5 kDa. However, the presence of up to three N-glycans increases the actual molecular weight of the protein with several kDa (depending on the N-glycans attached). Production in P. pastoris yields several hundred mg of mIL-22. It has also been possible to purify ˜100 mg/l of this cytokine from a mIL-22-producing M5 strain.

For each of the three proteins, five glycoengineered strains were constructed: M5, GnM5, GalGnM5, GalGnM3 and Gal2Gn2M3, according to the protocol outlined above. All introduced glycosylation genes were controlled by the constitutive GAP promoter. Nevertheless, most GlycoSwitch vectors exist in an AOX1 version as well.

### Mouse Interleukin 10 (IL-10)

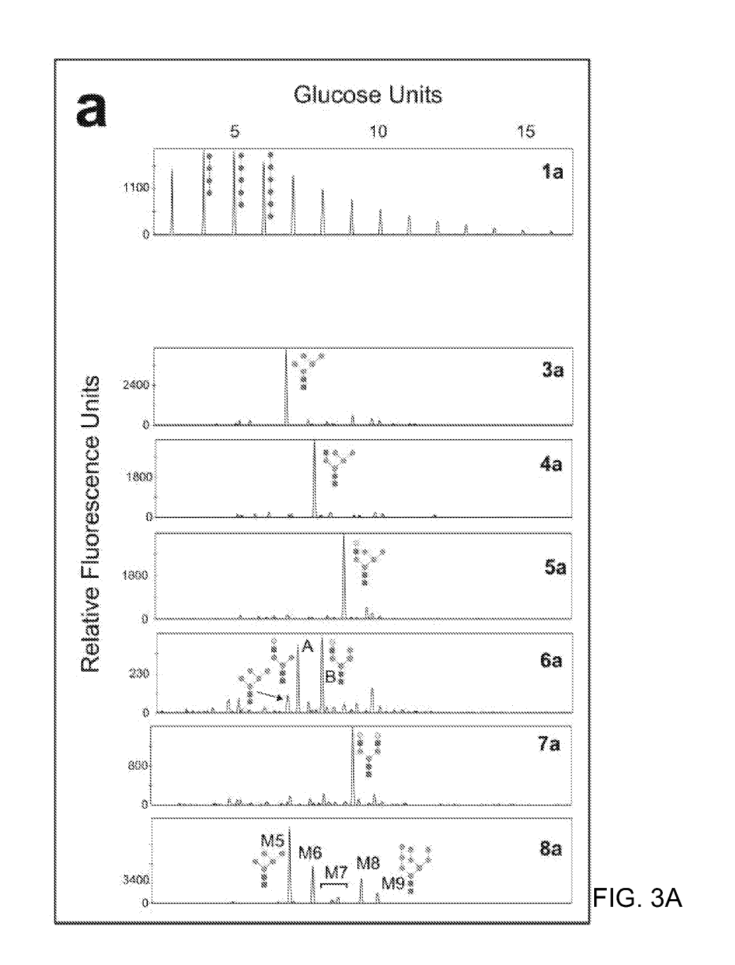

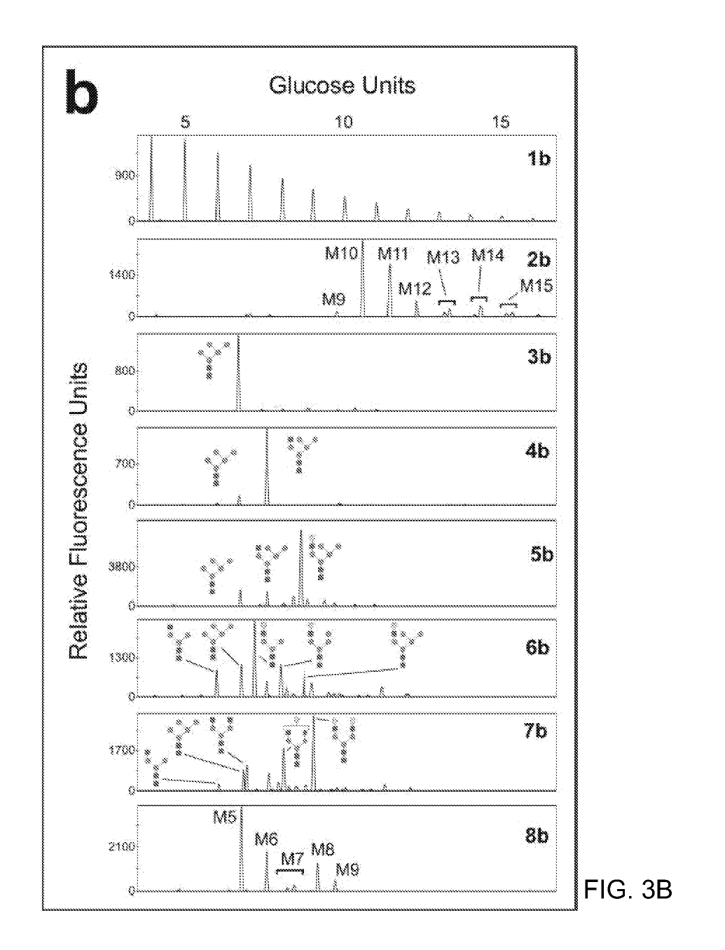

The DSA-FACE N-glycan profiles of the proteins present in unpurified growth medium after small-scale cultivation of the mIL-10-producing strains (Steps 23-29) are shown in FIG. 3a. DSA-FACE N-glycan analysis on mIL-10 purified from 250 ml shake flask cultures is shown in FIG. 3b. In Electropherogram 2b (FIG. 3b), the N-glycan profile of GS115-produced mIL-10 is shown. The predominant peaks are Man₁₀GlcNAc₂ and Man₁₁GlcNAc₂. Very large N-glycan species are often difficult to detect by DSA-FACE, because the high resolving power separates the myriad of isomers, which each have very low abundance. Therefore, N-glycan species larger than 20 residues are hard to detect by DSA-FACE (this also holds true for routine MALDI-TOF MS because of poor ionization efficiency of high-MW glycans). Electropherogram 3a (FIG. 3a) shows the total N-glycan pool on medium proteins from a MSmIL-10 culture. Electropherogram 3b (FIG. 3b) shows the N-glycans present on mIL-10 produced by this strain. Disruption of OCH1 and simultaneous overexpression of an ER-targeted α-1,2-mannosidase efficiently abolished hyperglycosylation and reduces the N-glycan pool to one predominant species, Man₅GlcNAc₂. Electropherogram 4a (FIG. 3a) shows the total N-glycan pool on medium proteins from a GnMSmIL-10 culture. Electropherogram 4b (FIG. 3b) shows the N-glycans attached to purified GnM5mL-10. The main peak is GlcNAcMan₅GlcNAc₂. A small fraction of Man₅GlcNAc₂, however, was not modified with a terminal GlcNAc residue. The effect of the introduction of a galactosyltransferase is shown in Electropherogram 5a and 5b (FIGS. 3a and 3b). In both profiles the predominant peak is GalGlcNAcMan₅GlcNAc₂. However, small amounts of GlcNAcMan₅GlcNAc₂ and Man₅GlcNAc₂ are present on the purified protein. Electropherogram 6a (FIG. 3a) shows the total N-glycan pool on medium proteins from a GalGnM3mL-10 culture. Electropherogram 6b (FIG. 3b) shows the N-glycan profile of mIL-10 produced in the GalGnMan3 strain. The observed heterogeneity is due to 1) incomplete processing of several intermediate structures (the peaks corresponding to Man₅GlcNAc₂, GalGlcNAcMan₅GlcNAc₂, and GlcNAcMan₃GlcNAc₂ in Electropherogram 6b of FIG. 3b are the result of non-quantitative substrate-to-product conversion by GnT-I, Man-II and GalT, respectively), and 2) the synthesis of N-glycan intermediates that can also serve as substrates for endogenous glycosyltransferases. It seems that GalGlcNAcMan₃GlcNAc₂ can be modified by one or more endogenous α-1,2-mannosyltransferases resulting in GalGlcNAcMan₄GlcNAc₂ (as was shown by in vitro treatment with α-1,2-mannosidase). The most efficient solution to this problem would be to identify the glycosyltransferases responsible and knock them out. With the availability of the P. pastoris genome this should be feasible. Alternatively, correctly sub-Golgi localized GnT-II is able to prevent the addition of an additional α-1,2-linked mannose residue by competing with the endogenous glycosyltransferase for the same N-glycan structure, GlcNAcMan₃GlcNAc₂ (FIGS. 3a and 3b, compare Electropherograms 6a and 7a). The N-glycan profile of mIL-10 produced in the most heavily engineered strain, Gal2Gn2Man3mL-10, is shown in Electropherogram 7a and 7b. The predominant peak is Gal₂GlcNAc₂Man₃GlcNAc₂. Galactosylation, however, is incomplete as evidenced by the presence of Gal₁GlcNAc₂Man₃GlcNAc₂ and GlcNAc₂Man₃GlcNAc₂ N-glycans. Additional in vitro polishing steps may be performed to achieve >90% homogeneous N-glycan profiles, as has been reported by others (Choi et al. Glycoconj J. 25: 581-593, 2008; Li et al., Nat. Biotechnol. 24: 210-215, 2006).

mGM-CSF and mIL-22

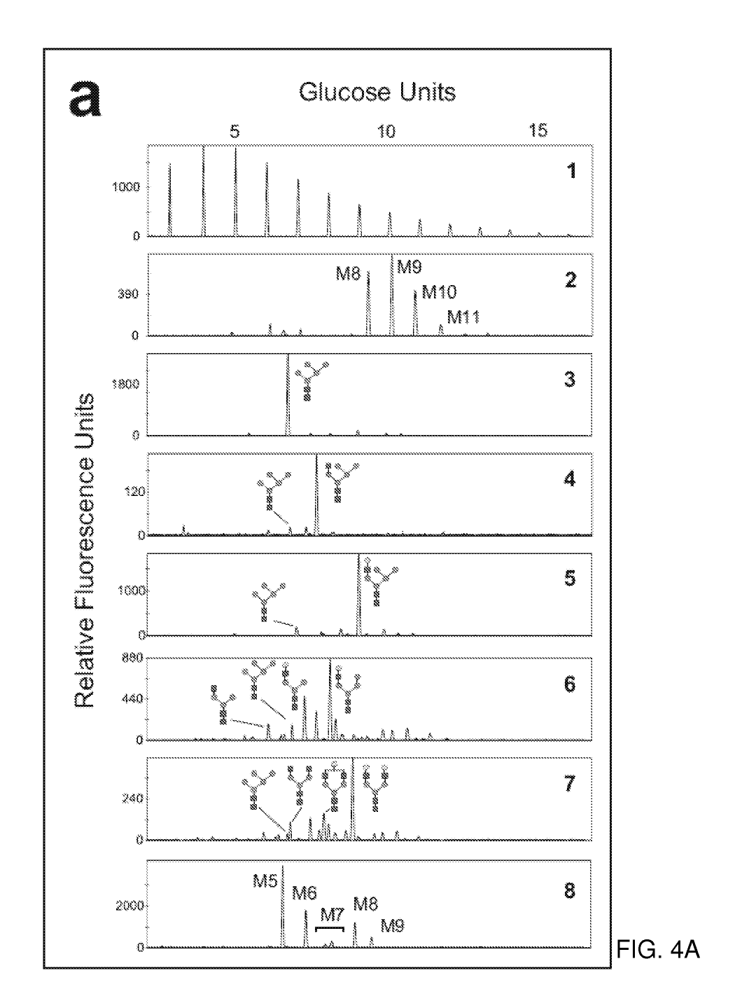

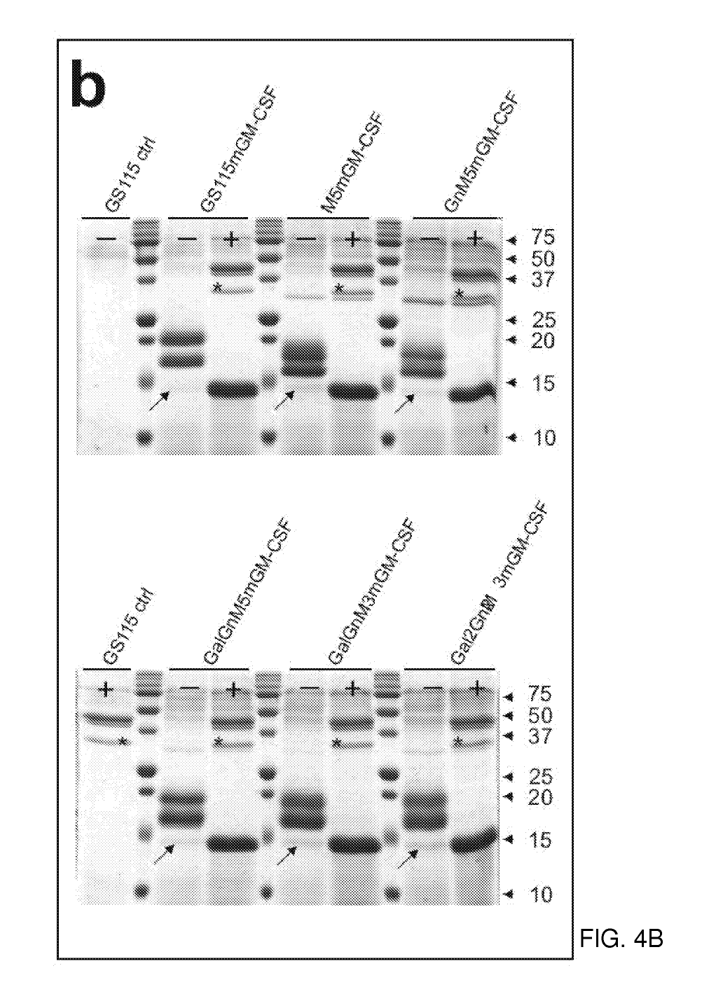

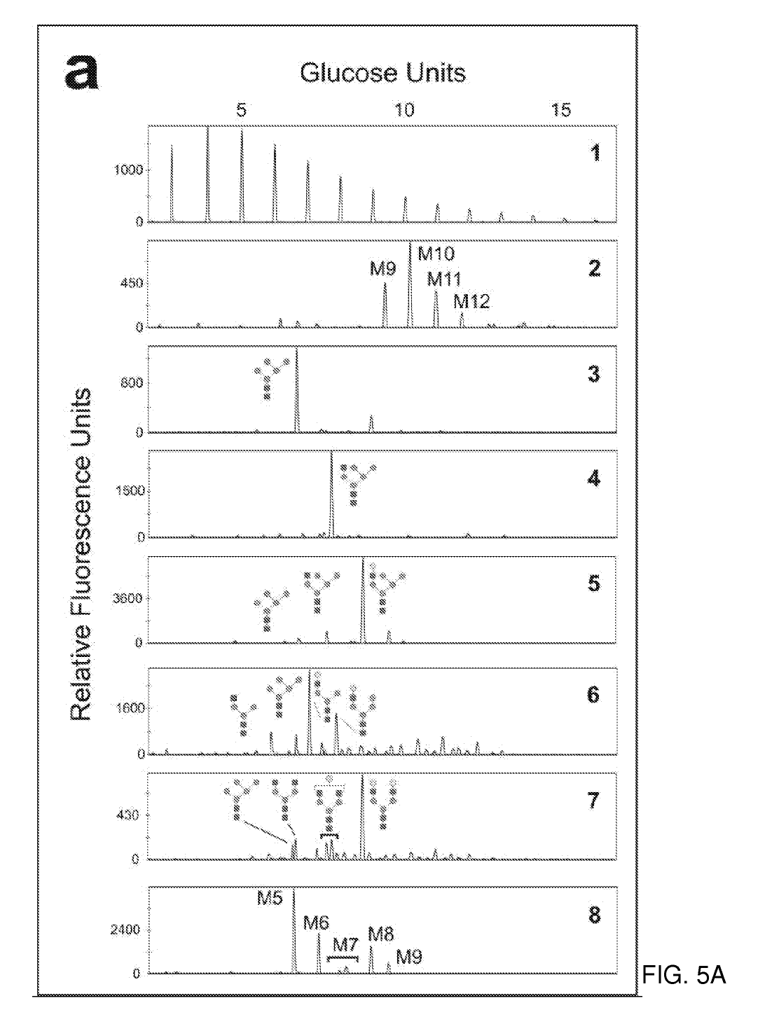

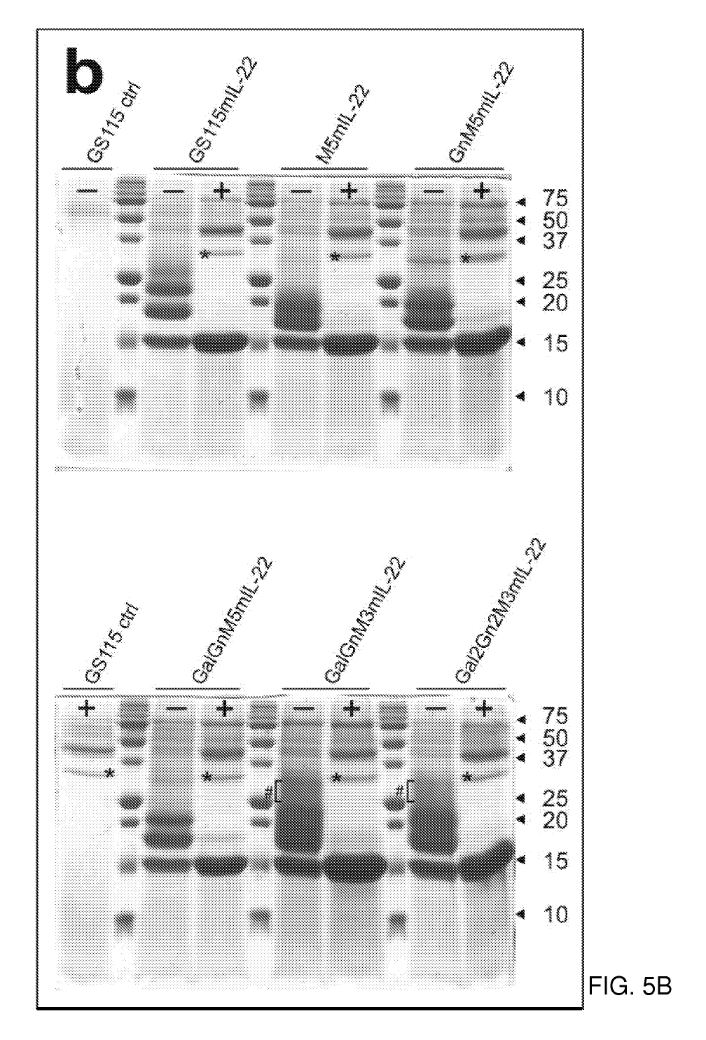

As was shown for mIL-10, mGM-CSF-producing and mIL-22-producing M5, GnM5 and GalGnM5 strains produce relatively homogeneous N-glycan profiles consisting predominantly of Man₅GlcNAc₂, GlcNAcMan₅GlcNAc₂, and GalGlcNAcMan₅GlcNAc₂ structures, respectively (FIGS. 4a and 5a). However, introduction of Man-II and GnT-II resulted in more heterogeneous N-glycan profiles. The GalGnM3mGM-CSF and Gal2Gn2M3mGM-CSF strains do produce GalGlcNAcMan₃GlcNAc₂ and Gal₂GlcNAc₂Man₃GlcNAc₂ as the predominant N-glycans, respectively, but a significant portion of their N-glycomes was composed of intermediate and high mannose structures (FIGS. 4a and 5a). This was due to interfering endogenous mannosyltransferases and non-quantitative conversion, mainly by GalT and to a minor extent by Man-II and GnT-II. Man-II seemed to have produced oligosaccharides that were substrates for endogenous glycosyltransferases implicated in outer chain synthesis. Especially in the case of mIL-22 this caused some smearing on SDS-PAGE (FIG. 5b). As can be concluded from FIGS. 4b and 5b, the glycoengineering process had no severe negative effect on mGM-CSF and mIL-22 yields.

### CONCLUSION

These results indicate that the more extensively engineered strains produce a relatively more heterogeneous array of glycoforms due to 1) incomplete processing of several intermediate N-glycan species and 2) some interference by endogenous mannosyltransferases. Both phenomena appear to be strongly influenced by growth conditions (compare FIG. 3a with FIG. 3b). Fermentation conditions can be further optimized to retain the more homogeneous N-glycan profiles, obtained in small-scale culture, after upscaling.

1. A method of producing a heterologous protein containing an Asn-X-Ser/Thr consensus N-glycosylation motif in Pichia, comprising
providing an auxotrophic Pichia strain whose genomic OCH1 gene has been inactivated, wherein said strain expresses said heterologous protein;
providing a series of vectors, each vector coding for one glycosylation enzyme selected from the group consisting of α-1,2-mannosidase (Man-I), N-acetylglucosaminyltransferase (GnT-I), β-1,4-galactosyltransferase (GalT), α-1,3/6 mannosidase (Man-II), and β-1,2-N-acetylglucosaminyltransferase (GnT-II), wherein said glycosylation enzyme is engineered to contain a signal that localizes said enzyme to the ER or the Golgi apparatus;
obtaining a Pichia clone that produces said heterologous protein bearing a predominant N-glycan structure, wherein said N-glycan structure is selected from the group consisting of M5 (Man₅GlcNAc₂), GnM5 (GlcNAcMan₅GlcNAc₂), GalGnM5 (GalGlcNAcMan₅GlcNAc₂), GalGnM3 (GalGlcNAcMan₃GlcNAc₂), GnM3 (GlcNAcMan₃GlcNAc₂), Gn2M3 (GlcNAc₂Man₃GlcNAc₂), and Gal2Gn2M3 (Gal₂GlcNAc₂Man₃GlcNAc₂), and wherein said clone is obtained by introducing into the Pichia strain of step a with one or more of said vectors in a sequential manner, wherein the introduction of each vector comprises transformation, cultivation of at least 10 transformants in small scale liquid cultures, analysis of N-glycans of glycoproteins and expression of said heterologous protein produced from each of said at least 10 transformants, and selection of a clone based on said analysis.

2. The method of claim 1, wherein a Pichia clone is selected after introduction of each vector that produces in a small-scale liquid culture said heterologous protein substantially homogenous in its N-glycan structure.

3. The method of claim 1, wherein said N-glycan structure is GalGnM3 (hybrid type) or Gal2Gn2M3 (complex-type).

4. The method of claim 1, wherein at least 20 transformants were cultivated for analysis and selection for introduction of each vector.

5. The method of claim 1, wherein said N-glycan analysis is done by way of DSA-FACE.

6. The method of claim 4, wherein said N-glycan analysis is done by using glycoproteins in a cell wall extract or in the culture medium.

7-10. (canceled)

11. A preparation of a heterologous protein made by any one of the methods of claims 1-6.

12-16. (canceled)


SEQUENCE LISTING (2 sequences)

SEQ ID NO: 1 - protein, 4 aa, Artificial Sequence
  Synthetic peptide localization sequence
HDEL

SEQ ID NO: 2 - protein, 4 aa, Artificial sequence
  Synthetic peptide localization sequence
KDEL